(19) United States
(12) Patent Application Publication
(10) Pub. No.: US 2012/0198910 A1
(43) Pub. Date: Aug. 9, 2012

(54) Breath Alcohol Analyser And A Method Of Determining Whether A Driver Is In A Condition To Drive

(75) Inventor: Kwok Chuen LO, Tsuen Wan (HK)

(73) Assignee: WEALTHYCOME INTERNATIONAL LIMITED, Tsuen Wan (HK)

(21) Appl. No.: 13/310,882

(22) Filed: Dec. 5, 2011

(30) Foreign Application Priority Data
Feb. 9, 2011 (HK) 11101239.6

(51) Int. Cl. G01N 33/497

(52) U.S. Cl. 73/23.3

(57) ABSTRACT

A breath alcohol analyser capable of taking a breath sample from a driver, qualitatively and quantitatively analysing the alcohol level in the driver's breath, comparing the alcohol to an established acceptable level for driving. If the alcohol level in the breath of the driver exceeds the acceptable level for driving, the breath alcohol analyser is capable of indicating to the driver how long need the driver wait in order for the alcohol in his body to breakdown to an acceptable level.

### FIELD OF THE INVENTION

The present invention relates to the field of analysers for detecting alcohol level in human breath.

### BACKGROUND OF THE INVENTION

Driving under the influence of alcohol is one known cause of traffic accidents. To discourage drivers from driving after drinking alcohol, traffic police tend to perform spot-checks on drivers on days when there is likely widespread alcohol consumption, such as holidays. Such spot-checks have to be on-the-spot and require instant detection. Thus, it is not possible to safely and efficiently rely on invasive test methods such as blood tests. Accordingly, the spot-checks are done by breath alcohol analysers which measure the amount of alcohol in the breath of the drivers, which is indicative of alcohol level in the driver's blood.

The breath alcohol analyser is a portable hand-held device which typically looks like an asthma inhaler. FIG. 1 shows a typical breath alcohol analyser comprising an exhalation nozzle 101 and an alcohol level sensor 102. The cross section of the exhalation nozzle 101 is round.

The driver has to blow into a mouthpiece of the breath alcohol analyser in order to provide a breath sample to be analysed for the alcohol content in the driver's breath. Within the breath alcohol analyser is a detector which can qualitatively and quantitatively measures alcohol in human breath. There are typically two types of such detectors, namely, the fuel cell type (electrochemical type) and the semiconductor type detector. In either case, the detector simply breaks down the alcohol, which in the process generates a voltage indicative of the amount of alcohol in the breath.

Typically, in order that breath taken from the driver is representative of the alcohol level in his body, the amount and flow rate of the breath should meet at least the following conditions:
(1) the breath exhaled should last continuously for 5 sec;
(2) the air pressure of the breath should be at least 12 mbar;
(3) the overall volume should be 0.5 ml; and
(4) the air velocity should be at least 33 m/s.

Sometimes, the breath of the driver tends to leak from the edges of his lips when he breathes into the mouthpiece, resulting in a failure to meet the foresaid requirement which leads to inaccurate analysis. Furthermore, the typical breath alcohol analyser is strenuous to use for elderly drivers, who are easily short of breath and need several attempts to meet the foresaid testing conditions.

Such portable breath alcohol analyser may be used by the driver himself to self-monitor his alcohol level may purchase his own analyser and monitor his own blood alcohol level voluntarily, before driving. However, it is common knowledge that alcohol intoxication could create a false sense of confidence. Under influence of alcohol, even if the breath alcohol analyser indicates that the alcohol level is higher than that officially allowed, the driver is unlikely to have enough self-control to restrain himself from driving, or be patient enough to wear off the alcohol.

Accordingly, it is desirable to propose suitable improvements for reducing or mitigating the described disadvantages.

### SUMMARY OF THE INVENTION

One of the objects of the present invention is to provide a breath alcohol analyser to detect the alcohol level in human breath and the time needed for reducing the alcohol level to the legal alcohol level acceptable for driving.

In a first aspect, the invention proposes a method of determining whether a driver is in a condition to drive comprising the steps of: sampling the driver's breath, determining the level of alcohol in his breath, determining the breakdown-time required to breakdown the alcohol in the driver's body to an acceptable level, and indicating to the breakdown-time.

Advantageously, through calculating the time needed for the alcohol to breakdown to an acceptable level in the driver's body, the traffic police, the driver or anyone looking after the driver may estimate the time by which the driver is likely to be able to drive. This allows the driver and anyone in his company to relax and do other things while he waits for the level of alcohol in him to breakdown. Furthermore, this allows the driver a sense of control as he is aware of how long he has to wait, which encourages the driver to patiently wear off the alcohol content. Alternatively, if the driver is unwilling to wait for the alcohol to breakdown in his body, he can choose to request someone else to be the driver. In this way, knowing of how long would the driver need to breakdown the alcohol in his body empowers the driver to make a safe decision with regards to driving.

Preferably, the breakdown-time is announced audibly. This advantageously increases social transparency and allows everyone in the location to hear and be alerted to when the driver is likely to be able to drive.

Preferably, the method further comprises a step of indicating a clearance alarm after the breakdown-time has passed, to indicate to the driver that he should be now cleared to drive. Thus, the driver need only wait for the clearance alarm instead of monitoring time himself. Optionally, the clearance alarm is an audible one. Alternatively, the clearance alarm is a visible one, such as flashing lights or LEDs, or a readable message in an LCD panel. The LEDs can colour coded to show green if the driver should be able to drive, and red to indicate that the driver should not be able to drive.

In a second aspect, the invention proposes a breath alcohol analyser comprising a sampler for taking the breath of a driver, an alcohol detector for determining the level of alcohol in the breath, a micro controller unit for calculating the breakdown-time required for the level alcohol to breakdown to a limit suitable for the driver to drive, an indicator to indicate the breakdown-time.

Preferably, the breath alcohol analyser further comprises a nozzle for the driver to blow into which has a cross-section of an oval shape. The oval cross-section allows a better fit between the human mouth and the nozzle, reducing the amount of leaked breath. This is especially advantageous for drivers who tend to be short of breadth, such as elderly derivers.

Preferably, the breath alcohol analyser has a memory storing the acceptable level of detectable alcohol in drivers; and further comprising a warning alarm, such as buzzer, which sounds if the alcohol level in the driver's breath is above the acceptable level of alcohol.

Preferably, the acceptable level of alcohol level can be set by the driver. Advantageously, this allows the breath alcohol analyser to be used in any country. Furthermore, the driver can even set a lower limit than the legal limit for increased safety when he drives.

Preferably, the breakdown-time is indicated audibly.

Preferably, a clearance alarm is raised after the breakdown-time has passed. More preferably, the clearance alarm is an audible alarm or the clearance alarm is a visible alarm. Possibly, the visible alarm is a readable indication of the breakdown-time.

In a third aspect, the invention proposes a breath alcohol analyser comprises an alcohol level sensor, a filter, a data sampling unit, a micro controller unit (MCU), an LCD driver and an LCD, wherein the output end of the alcohol level sensor is electrically connected to the input end of the filter, the output end of the filter is electrically connected to the input end of the data sampling unit, the output end of the data sampling unit is electrically connected to the input end of the MCU, and the output end of the MCU is electrically connected to the LCD with the LCD driver; the alcohol level sensor is used to detect alcohol level in human breath and output a voltage signal to the filter; the filter is used to filter the voltage signal; the data sampling unit is used to sample the voltage signal filtered by the filter and output the sampled voltage signal to the MCU; the MCU calculates the time t needed for reducing the alcohol level a to the legal alcohol level acceptable for driving b based on the sampled voltage signal, and outputs the time t; the LCD is used to display alcohol level a and time t and whether the alcohol level is acceptable.

t=(a−b)/k (1)
where
k is a coefficient;
a is the alcohol level
b is the legal alcohol level acceptable for driving;
t is the time needed to breakdown the alcohol from a to b

In a third aspect, the invention proposes a breath alcohol analyser comprising a nozzle for a driver to blow into, the nozzle having a cross-section of an oval shape. As mentioned, oval cross-section allows a better fit between the human mouth and the nozzle, reducing the amount of leaked breath. This is especially advantageous for drivers who tend to be short of breadth, such as elderly derivers.

Preferably, the breath alcohol analyser also comprises an auxiliary operational memory, which is used to store alcohol concentration a and time t.

Preferably, the standard alcohol concentration b may be set or adjusted by the driver.

Preferably, the breath alcohol analyser also comprises a power module, which is used to supply power for the breath alcohol analyser.

Preferably, the breath alcohol analyser also comprises an USB interface, which is electrically connected to the MCU with a data bus.

### BRIEF DESCRIPTION OF THE DRAWINGS

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 is a schematic diagram of a prior art;

FIG. 2 is a schematic of the circuit of an embodiment of the invention;

FIG. 3 is a schematic diagram of the embodiment of FIG. 2;

FIG. 4 is a illustrates the structure of the embodiment of FIG. 2; and

FIG. 5 is a schematic diagram of the power module of the embodiment of FIG. 2.

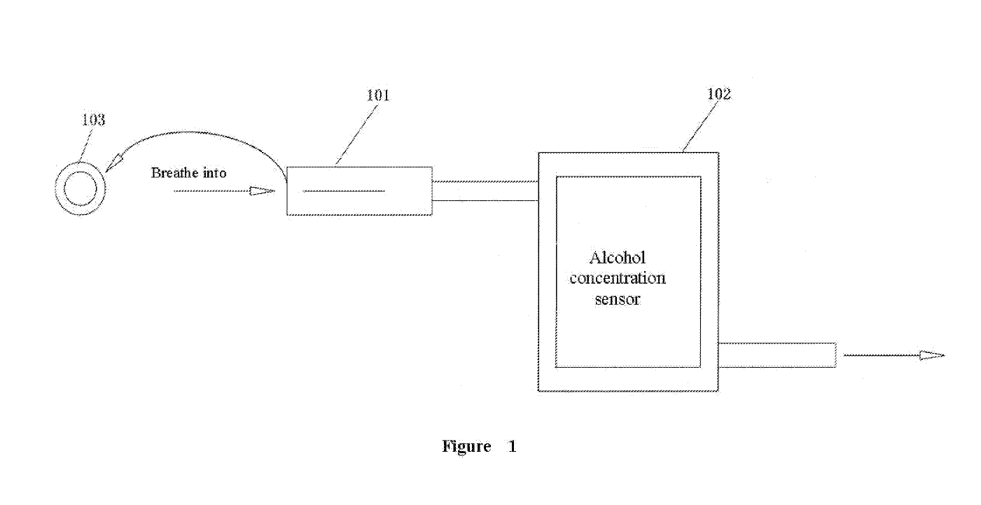

### DETAILED DESCRIPTION OF THE EMBODIMENTS

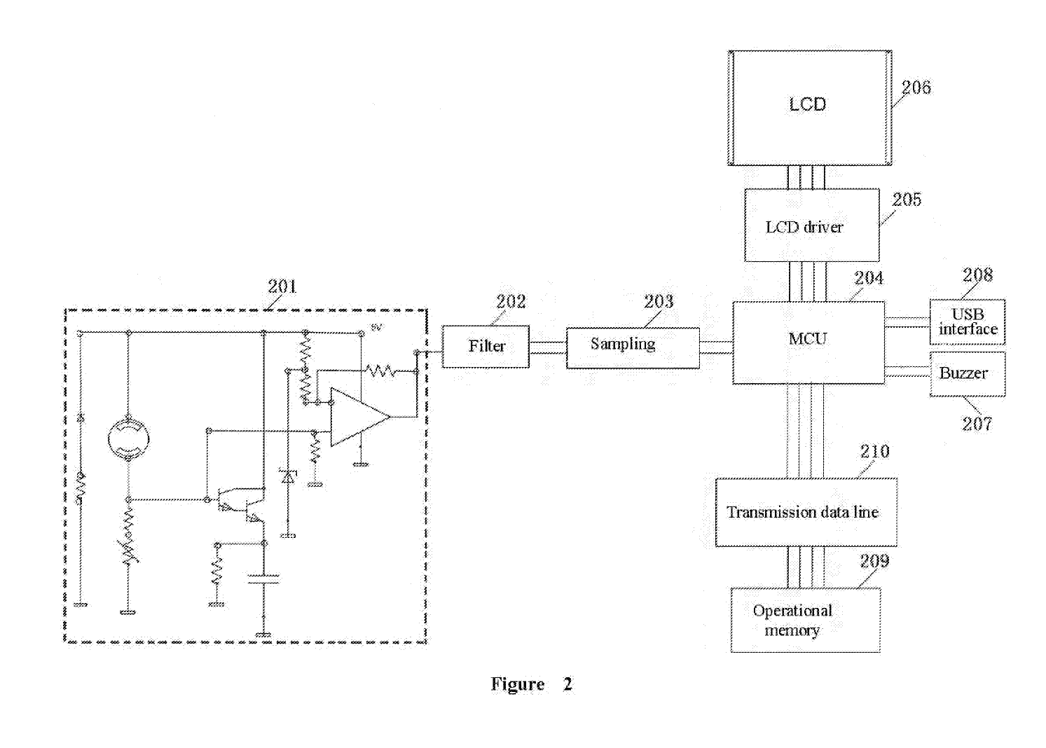

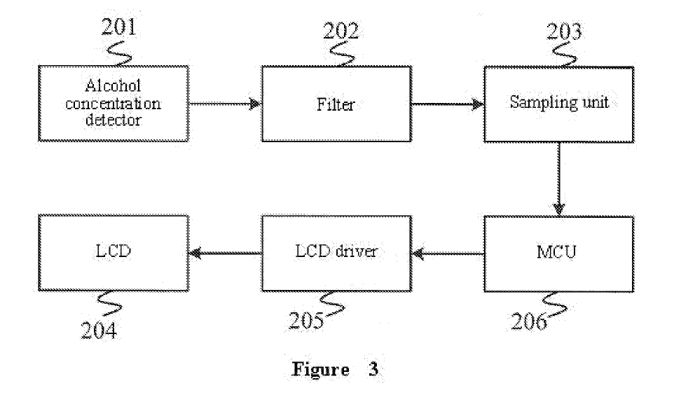

FIG. 2 is a circuit schematic of a breath alcohol analyser and FIG. 3 is an illustration of the different functional blocks within the breath alcohol analyser. Typically, the outer appearance of this embodiment is like a portable asthma inhaler (not illustrated).

Within the breath alcohol analyser is an alcohol detector 201, a signal filter 202, a data sampling unit 203, a micro controller unit (MCU) 204, an LCD 206 driver 205 and an LCD 206.

Typically, the alcohol detector 201 is a fuel cell type alcohol detector, and detects alcohol level in human breath and output a voltage signal. The fuel cell alcohol detector comprises platinum electrodes and a combustion chamber filled with a suitable catalyst as is known in the art. Thus, the catalyst requires no further elaboration. The catalyst causes alcohol carried in human breath passing through the combustion chamber to ‘burn’ or breakdown, which generates electric energy detected as a voltage between two electrodes. The magnitude of the voltage is in direct proportion to the alcohol level in the breath; the higher the alcohol level the higher the voltage.

The voltage between two electrodes is fed as a signal into the signal filter 202 to filter away noise in the signal. Subsequently, signal filter 202 output is fed to the data sampling unit 203, which samples the voltage digitally, converting the voltage signal into digital data. The data sampling unit 203 then sends the digital data to the MCU 204. In this way, the data sampling unit 203 detects the waveform of the signal filter 202 output, extracts a sample waveform and outputs it to the MCU 204.

The MCU 204 calculates the alcohol level from the digital data and. Furthermore, the MCU 204 calculates the time required for the alcohol level to break down to an acceptable level for driving, i.e. breakdown-time. The algorithm, equation or software, and any statistics for the calculation may have already been stored in a memory in the breath alcohol analyser.

An example of an equation is one which may be based on known human efficiency for breaking down alcohol is shown as Equation (1). This may be obtained from textbooks, or with the help of professionals or academics in the appropriate fields and requires no elaboration here.

t=(a−b)/k (1)
where
k is a coefficient;
a is the alcohol level
b is the legal alcohol level acceptable for driving;
t is the time needed to breakdown the alcohol from a to b.

Typically, the average constant of the metabolic rate of breath alcohol level is 0.1 mg/L/hr. Thus, k is typically 0.1. Accordingly, if a driver's measured alcohol level a=0.8 mg/L, and the legal alcohol level acceptable for driving, b=0.2 mg/L, then t=(0.8−0.2)/0.1=6 (h). In other words, the driver's alcohol level may be reduced to below the legal alcohol level acceptable for driving six hours later.

The MCU 204 and transmits the alcohol level and the breakdown-time to the LCD 206 driver 205 to be displayed. The breath alcohol analyser further comprises an USB interface, which is electrically connected to the MCU 204 with a databus, and may be used to connect to other suitable computing breath alcohol analysers, any peripheral device connected to the MCU such as a printer or a network connection The MCU 204 may also used to sequence data and perform I/O operation.

The output end of the MCU 204 is electrically connected to the LCD 206 with the LCD 206 driver 205. The LCD 206 displays alcohol level a and time t and whether the breath alcohol level is legally acceptable for driving. Through calculating the time needed for reducing the alcohol level to the legal alcohol level acceptable for driving, the driver may know when he/she may start driving.

Generally, the value of b may be pre-set in the factory according to local traffic laws or health regulations. Optionally, the legal alcohol level acceptable for driving b may be set by the driver. This allows the driver to set the standard according to the country he is in, or to set a stricter standard for safer driving for himself. For example, if the value displayed is 0.4 mg/L when somebody does a breath test in Japan, and suppose that the Japanese standard value is 0.25 mg/L, the time t of 2 hours will be displayed as the breakdown-time. That means that the driver will be able to start driving legally two hours later.

The specific formula may be varied as would be known to the skilled man. For example, the unit of alcohol level may be % BA, mg/L or % BrAc. Due to different standards in different countries, it is an optional feature that the driver may select the unit for calculation. Alternatively, instead of a formula, a database of statistically collected data may be used to estimate the alcohol breakdown time according to the to alcohol level, a.

Typically, the fuel cell alcohol detector, the data sampling unit 203 and the MCU 204 have been factory-calibrated in such a way that the breath alcohol analyser may be used without need of re-calibration by any user or driver. For example, the coefficients of calculating the alcohol level form the voltage may have been determined and stored in the breath alcohol analyser memory.

Preferably, the breath alcohol analyser comprises a buzzer, which is electrically connected to the MCU 204 and which issues an alarm if the detected alcohol level is above the legal limit.

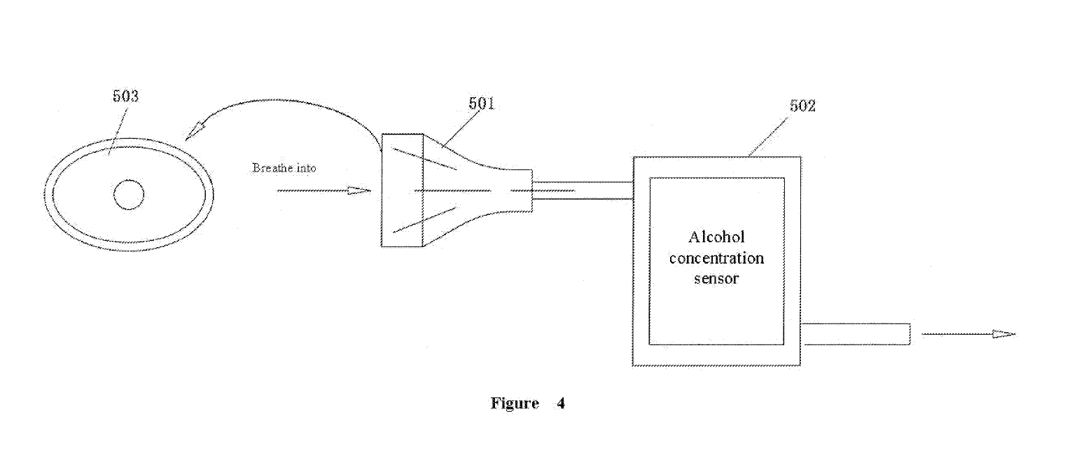

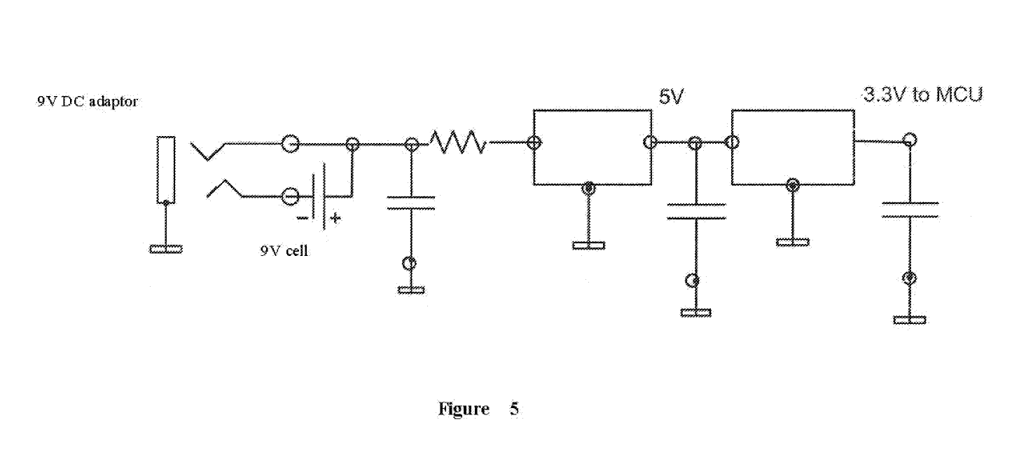

Preferably, as shown in FIG. 4, the cross-section 503 of the exhalation nozzle 501 is in an oval shape. Compared to a round shape, the oval shape is likely to have less leakage of the breath breathed into the breath alcohol analyser, which increases the amount of breath passing through the breath alcohol analyser by even ten folds over the round shape nozzle, improve the accuracy of the breath alcohol analyser.

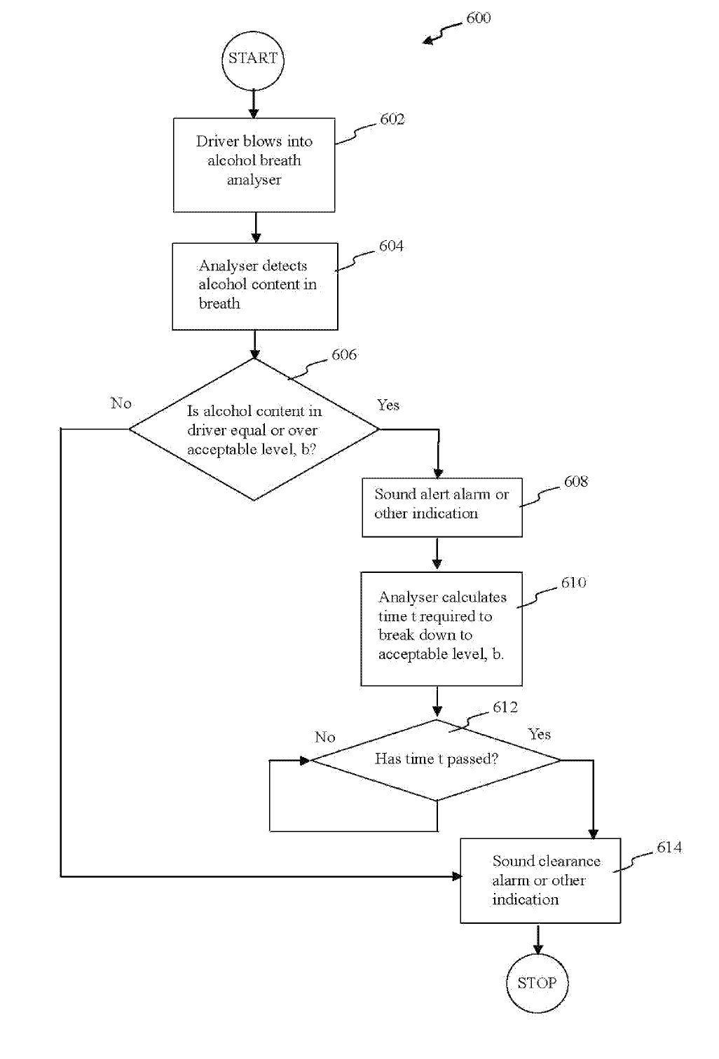

FIG. 5 illustrates the breath alcohol analyser comprising a power module, such as a rechargeable battery, which supplies power to the functional modules in the breath alcohol analyser. For example, +5 V is supplied to the fuel cell type detector, and +3.3 V is supplied to the signal filter 202, the data sampling unit 203, the MCU 204, the LCD 206, the memory and etc. An operational amplifier may also be provided in some embodiments which adjusts the voltage level to any module, as required.

FIG. 6 is a flowchart 600 illustrating the steps in the embodiment for calculating and indicating whether a driver's alcohol level is legally acceptable for driving. In the beginning, the driver blows into the mouthpiece of the breath alcohol analyser, at step 602. The breath alcohol analyser then detects the alcohol content in the driver's breath, at step 604. If the alcohol content in the driver's breath is below the acceptable lever, b, the breath alcohol analyser indicates to the driver that he is cleared to drive, at step 614. The indication can be by means of an audible alarm or recording, flashing LEDS lights or digital display of a readable message. If the alcohol content in the driver's breath is above or equal to the acceptable lever, b, the breath alcohol analyser indicates to the driver that he is not cleared to drive. The indication can be by means of an audible alarm or recording, flashing LEDS lights or digital display of a readable message, at step 608. The breath alcohol analyser calculates the time, t, which the driver's alcohol content is likely to breakdown into to acceptable level, b, at step 610. The breath alcohol analyser then monitors the time, at step 612. Once the breath alcohol analyser detects that time, at step 612, has passed, the breath alcohol analyser indicates to the driver that he is cleared to drive, at step 614.

Preferably, the MCU in the breath alcohol analyser may be programmed to remind the driver to provide another sample of this breath after the breakdown-time has passed, in order to ensure that the driver is really in a safe condition to drive.

Details such as how to calibrate the breath alcohol analyser or how to ensure that the flow rate of the exhaled breath from the driver is useable to indicate the alcohol level is part of known quantification technology and need no elaboration here.

Although the afore-described embodiments indicate that the breakdown time may be calculated and indicated to the driver, it is understood that variations due to factors in practical situations may be expected, and the indication is usually just a good estimate and not a fixed.

Therefore, the embodiment comprises a method of determining whether a driver is in a condition to drive comprising the steps of: sampling the driver's breath; determining the level of alcohol in his breath; determining the breakdown-time required to breakdown the alcohol in the driver's body to an acceptable level; and indicating to the breakdown-time.

Furthermore, the embodiment is a breath alcohol analyser comprising a nozzle for taking the breath of a driver; alcohol detector 201 for determining the level of alcohol in the breath; a micro controller unit for calculating the breakdown-time required for the level alcohol to breakdown to a limit suitable for the driver to drive; an indicator to indicate the breakdown-time.

More specifically, the embodiment comprises a breath alcohol analyser comprises an alcohol level sensor, a signal filter 202, a data sampling unit 203, a micro controller unit (MCU 204), an LCD 206 driver 205 and an LCD 206, wherein the output end of the alcohol level sensor is electrically connected to the input end of the signal filter 202, the output end of the signal filter 202 is electrically connected to the input end of the data sampling unit 203, the output end of the data sampling unit 203 is electrically connected to the input end of the MCU 204, and the output end of the MCU 204 is electrically connected to the LCD 206 with the LCD 206 driver 205; the alcohol level sensor is used to detect alcohol level in human breath and output a voltage signal to the signal filter 202; the signal filter 202 is used to signal filter 202 the voltage signal; the data sampling unit 203 is used to sample the voltage signal filter 202ed by the signal filter 202 and output the sampled voltage signal to the MCU 204; the MCU 204 calculates the time t needed for reducing the alcohol level a to the legal alcohol level acceptable for driving b based on the sampled voltage signal, and outputs the time t; the LCD 206 is used to display alcohol level a and time t and whether the alcohol level is acceptable.

It should be understood that the foregoing descriptions are the embodiments of the present invention and not intended to limit the protection scope of the present invention, and all modifications, identical replacements and improvements made without departing from the spirit and principle of the present invention shall be within the protection scope of the present invention.

What is claimed is:

1. A method of determining whether a driver is in a condition to drive comprising the steps of:
sampling a driver's breath;
determining a level of alcohol in the driver's breath;
determining a breakdown-time required to breakdown the determined level of alcohol in the driver's body to an acceptable level; and
indicating the breakdown-time to the driver.

2. The method of determining whether a driver is in a condition to drive, as claimed in claim 1, wherein the breakdown-time is announced audibly.

3. The method of determining whether a driver is in a condition to drive, as claimed in claim 1, further comprising the step of:
generating a clearance alarm after the indicated time has passed.

4. The method of determining whether a driver is in a condition to drive, as claimed in claim 3, wherein the clearance alarm is an audible alarm.

5. The method of determining whether a driver is in a condition to drive, as claimed in claim 3, wherein the clearance alarm is a visible alarm.

6. The method of determining whether a driver is in a condition to drive, as claimed in claim 5, wherein the visible alarm is a readable indication of the time.

7. A breath alcohol analyser comprising
a sampler for sampling a breath of a driver;
an alcohol detector for determining a level of alcohol in the breath;
a micro controller unit for calculating a breakdown-time required for the determined level of alcohol to breakdown to a limit suitable for the driver to drive;
an indicator to indicate the breakdown-time.

8. The breath alcohol analyser as claimed in claim 7, further comprising a memory storing an acceptable level of detectable alcohol in a driver's breath; and
a warning alarm for warning when the alcohol level in the driver's breath is equal to or above the acceptable level of detectable alcohol.

9. The breath alcohol analyser as claimed in claim 8, wherein the acceptable level of alcohol level can be set by the driver.

10. The breath alcohol analyser as claimed in claim 7, wherein
the breath alcohol analyser generates a reminder to request that the driver provide another sample for re-determining the level of alcohol in the breath after the breakdown-time has passed.

11. The breath alcohol analyser as claimed in claim 7, wherein
the breakdown-time is indicated audibly.

12. The breath alcohol analyser as claimed in claim 7, wherein
a clearance alarm is raised after the breakdown-time has passed.

13. The breath alcohol analyser as claimed in claim 12, wherein
the clearance alarm is an audible alarm.

14. The breath alcohol analyser as claimed in claim 12, wherein
the clearance alarm is a visible alarm.

15. The breath alcohol analyser as claimed in claim 12, wherein the visible alarm is a readable indication of the breakdown-time.

16. The breath alcohol analyser as claimed in 7 further comprising a nozzle for a driver to blow into, the nozzle having a cross-section of an oval shape.
